SEQUENCE LISTING (5 sequences)

SEQ ID NO: 1 - DNA, 588 bp, synthetic construct
  source 1..588: mol_type = other DNA
cacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtcca

SEQ ID NO: 2 - DNA, 588 bp, synthetic construct; identical to SEQ ID NO: 1
  source 1..588: mol_type = other DNA

SEQ ID NO: 3 - protein, 196 aa, synthetic construct
HLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACP

SEQ ID NO: 4 - DNA, 588 bp, synthetic construct
  source 1..588: mol_type = other DNA
cagcagtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtcca

SEQ ID NO: 5 - protein, 196 aa, synthetic construct
QQWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACP

(19) United States
(12) Patent Application Publication
(10) Pub. No.: US 2024/0350618 A1
(43) Pub. Date: Oct. 24, 2024

(54) ANTI-HIV-1 RECOMBINANT HIV-1 DERIVED TOPOISOMERASE II BETA KINASE AS AN IMMUNOGEN FOR HIV VACCINE

(71) Applicant: UNIVERSITY OF HYDERABAD, Hyderabad (IN)

(72) Inventors: Satyajit Mukhopadhyay, Hyderabad (IN); Anand Kumar Kondapi, Hyderabad (IN); Ritika Das, Hyderabad (IN); Miss Hema, Hyderabad (IN)

(21) Appl. No.: 18/443,332

(22) Filed: Feb. 16, 2024

(30) Foreign Application Priority Data
Feb. 28, 2023 (IN) 202341005688

(51) Int. Cl. A61K 39/21; A61K 39/00; C12N 7/00; C12N 9/90

(52) U.S. Cl. CPC A61K 39/21; C12N 7/00; C12N 9/90; C12Y 599/01002; A61K 2039/53; C12N 2740/16034

(57) ABSTRACT

The present invention relates to an anti-HIV recombinant HIV-1 derived Topoisomerase IIβ kinase. It inhibits HIV-1 replication by blocking viral entry. Its recognition by envelope antibodies ID6 and 4G10 makes it a justifiable immunogen for use as vaccine candidate in form of protein, mRNA and DNA vaccine against HIV infection. Thus, the protein, mRNA and DNA of immunogenic recombinant HIV-1 derived Topoisomerase IIβ kinase and derived peptides with and without spacers can be used as a HIV vaccine.

FIG. 1, FIG. 2, FIG. 3, FIG. 4, FIG. 5, FIG. 6, FIG. 7, FIG. 7, FIG. 8, FIG. 9, FIG. 10, FIG. 11, FIG. 12, FIG. 13, FIG. 14, FIG. 15, FIG. 16.

### FIELD OF INVENTION

The present invention relates to the field of a pharmaceutical composition containing a recombinant HIV-derived Topoisomerase II beta kinase and peptides derived from it as vaccine candidates, it is more specifically related to the field of anti-HIV vaccines that can be used for immunization for developing antibodies that can neutralize the virus with specifically binding to virus envelop protein for protection from HIV infection. Our present invention relates to an anti-HIV recombinant HIV-1 derived Topoisomerase IIβ kinase, a rec-TopoIIβK_{ENV-HIV} protein as an immunogen for HIV vaccine.

### REFERENCE TO SEQUENCE LISTING

The substitute sequence listing is submitted as an ASCII formatted text filed via EFS-Web, with a file name of “Substitute_Sequence_Listing_10-58-USUTIL.xml”, a creation date of Feb. 13, 2024, and a size of 8000 bytes. The substitute sequence Listing filed via EFS-Web is a part of the specification and is incorporated in its entirety by reference herein.

### BACKGROUND OF THE INVENTION

Human Immuno-deficiency Virus (HIV)-a species of Lentivirus (a subgroup of retrovirus) is a life-threatening virus transmitted via infected body fluids and causes the Acquired Immuno-Deficiency Syndrome (AIDS); It destroys the infected body's ability to defend itself and makes the person vulnerable to all kinds of infections. The virus targets the white blood cells (the CD4 positive T cells which provide immunity) in the blood. They enter these cells and replicate themselves and destroy the white blood cell. The nascent HIV virus target other CD4 positive cells and infection progresses in different cells and tissues, thereby destroying the immunity of the host body. While there are no medicines to eradicate the HIV from the body of infected person, many medicines and treatments exist to prevent the patient from developing AIDS by curtailing the viral load, preventing disease progression. Several efforts are in place for develop vaccines for protection of a person from HIV infection.

According to the WHO (2020), more than 37 million people are infected with HIV, the causative agent of AIDS. More than a disease, it is a debilitating factor on many levels including physical, socio-economical, cultural and others. Though many medications have been developed for the mitigation of sufferings by those affected by AIDS, the constraints of its high genomic variability make the development of HIV drugs a challenge.

An effective vaccine against HIV is the need of the hour. HIV vaccine development has been quite a bit of a challenge due to the high amount of variability in the viral genome, latent viral reservoirs at the early stages, evasion of the cellular and humoral immune response by the viral particles, and some un-explained immune regulation alterations. The vaccines introduced to date are either ineffective or with a negligible shelf-life.

The past few decades have witnessed several advances made in HIV vaccine development, including gene-based vaccines, among which only five could make their way to the human trials and only two have been licensed for animal use (fish and horse). In an NIH press release in 2017, it was experimentally depicted that HIV vaccine candidates provided insufficient protection to women (25.2%) against HIV infection.

A clinical trial in 2021 revealed that the infusion of broadly neutralizing antibodies-VRC01 can prove to be effective against HIV, though with a constraint of the strains which are susceptible to antibodies. mRNA vaccines paved the way toward HIV vaccine development when the first human trials of such mRNA-based vaccines were initiated in early 2022 in the United States. Gp 120 protein consists of five constant and five variable regions. Earlier these constant regions were principally targeted in terms of the generation of neutralizing antibodies against HIV-1. Recent studies show that the five variable regions show alterations in individuals, and specifically the V3 loop interacts with the host cellular CD4 receptor and thus has become an essential target for designing viral entry inhibitors. The current studies on entry inhibition majorly focus on the interaction of the V3 loop with the CD4 receptor. Studies have been conducted targeting the V2 loop of gp120 envelope protein in terms of vaccine development. Cyclically permuted gp120 (cycP gp-120) has been considered in this regard, to test its efficacy to generate neutralizing antibodies against HIV. Several studies based on drug designing aims majorly at the inhibition of the fusion reaction of the viral gp 120 and the cellular receptor CD4. This includes drugs that mimic the CD4 receptor, thus neutralizing the viral gp120 ligand Some others down-regulate the expression of the CD4 receptor at the post-translational level. Some are monoclonal antibodies that compete with gp 120 to bind to CD4 but its clinical application has the constraints of infusion. Others bind and block the conformational changes in gp120, which inhibits the viral entry into the host cell.

Though several studies have been conducted on HIV entry inhibitors, no such report has yet been published regarding this specific HIV protein, its binding position, and hence its role in entry and other functions, which would make it a good therapeutic and a vaccine candidate. HIV envelope plays a major role in viral entry into the host cell. Each envelope molecule (gp160) is a heterodimer of non-covalently linked gp120 and gp41, which after synthesis on the rER assembles as a trimer.

These trimers undergo some initial folding arrangements following which they are N-glycosylated (which provides a binding site for CD4) and transported to the plasma membrane via the Golgi apparatus. It is during this transport process that gp160 is proteolytically cleaved into gp 120 (extracellular) and gp41 (transmembrane). Gp120 contains five conserved and five variable domains, the latter is majorly located in the disulphide loop regions. Whereas gp41 has an ectodomain, which is essential for membrane fusion, the transmembrane region—for anchorage and the cytoplasmic tail. The binding of CD4 causes major structural changes in the trimer, which is normally held together by the V1/V2 loop regions. CD4 binding sites are generally recessed by about 20 angstroms top of the spike, thus only when the CD4 reaches deep into the spike, proper binding is achieved. Apart from CD4 which is the receptor, HIV uses certain co-receptors like CCR5 and CXCR4 for its entry into the cell. Viruses that are responsible for primary infections use CCR5 and are known as R5 HIVs, and those that cause later-stage infections use CXCR4 and are referred to as X4 HIV. All these receptors are expressed on the CD4+T lymphocytes and myeloid cells i.e. dendritic cells and macrophages. Thus, HIV primarily targets these CD4+ T cells, and after transmission, it reaches the mucosal tissues, which within a few days finally spreads to the lymphoid organs. The hinge region of CD4 shows a dramatic bend in its conformation on binding to gp120. The V3 loop also plays a major role in viral tropism and the choice of the chemokine receptor, and only in its presence gp 120 can bind to CCR5.

The structural change in gp120 aids in viral entry. The outward movement of this protein leads to a marked change in the two outermost domains-namely DID2 and D3D4 of membrane-anchored CD. This also pulls the viral and the cell membranes closer to each other and hence helps in fusion and entry. Thus, targeting this step of viral entry into the host cell would be beneficial in the combat against HIV.

HIV-1-derived Topoisomerase II β kinase (TopoIIβKHIV-I), a truncated N-terminal 196 amino acid protein, was identified in the lab work. This protein is involved in the phosphorylation of Topoisomerase II β, resulting in conformational changes in the protein promoting reverse transcription reaction. Recent studies have shown that this protein has been derived from the N-terminal portion of gp120.

Thus, current therapeutic approach focuses heavily on inhibiting pre-integration steps targeting key proteins involved in the replication cycle: envelope (ENV), reverse transcriptase (RT), integrase (IN), and protease (PR). The same is reflected in FDA approved list of administrable anti-HIV drugs consisting predominantly of fusion inhibitors, RT inhibitors (NRTIs and NNRTIs) and protease inhibitors. The recent advance in treatment has been the advent of integrase inhibitors entailing the prevention of viral replication.

Peptide based vaccine against HIV has been quite an interest in recent times due to the advantages that it bears as compared to other kinds. These include easy production, high stability, less of toxic substance, less complex antigens, and cost effectiveness.

A complete cure for HIV would entail both remission and eradication. The problems of drug resistant mutations along with viral genome diversity shaped the antiretroviral drug regime to daily administered combinational antiretroviral therapy (cART), which led to a significant drop in AIDS-related morbidities and mortality. Currently 28.7 million people were accessing antiretroviral therapy (ART) in 2021. Though cART improves the quality of life of HIV patients and drops the chance of transmission; its adverse side effects including premature aging, drug fatigue, toxicity, and inflammatory effects raises concern. These side effects exacerbate the incidence of other diseases, such as cardiovascular disease and chronic obstructive pulmonary disease (COPD).

Some of the patents related to our present invention have been discussed below: The patent EP2356133B1 (Recombinant protein bodies as immunogen-specific adjuvants) relates to a vaccine for use in a method for inducing T-cell mediated immune response against an immunogenic peptide, wherein the vaccine is a nucleic acid molecule that encodes a fusion protein, said recombinant fusion protein containing two portions peptide-linked together in which a first portion is a protein body-inducing sequence (PBIS) and a second portion is an immunogenic polypeptide.

The patent AU2004283288B2 (Immunogenic composition and method of developing a vaccine based on portions of the HIV matrix protein) relates to substance based on the amino terminal end of the matrix protein (p17MA) and covalent binding site for myristate of the HIV virus for achieving the same.

The HIV is an enveloped virus with a spherical capsid containing 72 knobs, each knob is composed of trimers of the Env proteins. The viral envelope protein composed of surface protein gp120 (SU) and transmembrane protein gp41 (TM). It covers the symmetrical outer capsid membrane which is formed by the matrix protein. The conical capsid is assembled from the inner capsid protein. The tapered pole of the capsid is attached to the outer capsid membrane. Two identical molecules of viral genomic RNA are located inside the capsid and several molecules of the viral enzymes RT/RNase H and IN bound to the nucleic acid. The virus contains regulatory proteins like Tat, Nef and Rev, Vif, Vpr and Vpu which influence the rate of the production of virus particles and perform different specific functions. HIV delivers its genome into the host cell cytoplasm in a series of complex steps while simultaneously evading the host immune response. To infect cells, the HIV protein envelope (Env) binds to the primary cellular receptor CD4 and then to a cellular coreceptor. This sequential binding triggers fusion of the viral and host cell membranes, initiating infection. This field is widely researched over the years for development of medicines and treatment of HIV. The current anti-retroviral therapeutics principally target viral integrase, reverse transcriptase, protease and envelope protein, responsible for the entry, infectivity and establishment of the virus infection. HIV-1 entry is mediated in CD4 receptor and CXCR4 or CCR5 co-receptors; their inhibitors analyzed for targeting HIV-1 infection. A direct correlation has been observed between the decrease in the efficiency of HIV1 replication and the depletion of Topoisomerase II (Topo II) level by the antisense oligonucleotide, SiRNA knockdown and poisoning of Topo II. SiRNA-mediated knockdown of Topo II isoforms shown to play a critical role in formation of intermediates in HIV-1 reverse transcription and HIV-1 tat mediated viral mRNA synthesis. Thus, Topo II isoforms could be of potential interest in targeting HIV-1 replication. Since Topo II isoforms are cellular proteins and required for maintenance of several housekeeping functions of cells, targeting these enzymes will be lethal to cell survival. Topo II isoforms are differentially phosphorylated by two fractions of purified HIV-1 lysate. A 72 kDa protein, HIV-1-derived Topoisomerase IIβ kinase (Topo IIβKENV-HIV), has been found to be present in second fraction of purified HIV-1 virus lysate. Topo IIβKENV-HIV is a Ser/Thr kinase (STK) and is shown to be resistant to a panel of 20 potent STK and tyrosine kinase inhibitors including staurosporine and PD98059, thus establishing Topo IIβKENV-HIV is a novel kinase that is expressed during HIV-1 infection and encapsulated into the virus. Since Topo IIβKENV-HIV is not found to be present in healthy uninfected T cells, it can form unique target for interfering HIV-1 replication in infected cells. In HIV-1 infected individuals, about one-third people develop broad neutralizing antibodies recognizing highly conserved region of viral antigens within five years post infection, thus contributing to controlling virus titers through effective neutralization. Many of these broad neutralizing antibodies recognize conserved epitopes of envelope involved in recognition of host receptors like CD4 and Co-receptors. These antibodies also mediate inflammatory responses to enhance immunity. Mostly glycan-based epitopes reported to play a role in effective neutralization of virus. Based on this concept BG505.SOSIP.664 gp140 was developed as immunogen based on clade A Env, which is under clinical trial (NCT03699241). Envelope lineage vaccines of envelope for B-cell activation were reported to exhibit broad neutralizing antibodies are in a clinical study (NCT03220724). The failure of vaccine could be due to the variations (35%) in antigenic regions in viral envelope along with development of escape mutants of virus evolution due to antigenic pressure. Thus, warrant development more vaccine candidates with potential to generate highly neutralizing antibodies.

Our present invention relates to the role of an N-terminal 72 KDa portion of gp120 in viral entry inhibition through a strong competition with virus for receptor binding, evaluate immunologic potential and other functions leading to virus neutralization, and evaluating its efficacy of rec-TopoIIβK_{ENV-HIV} as an immunogen in HIV vaccine.

### Objective of the Invention

The main objective of our present invention is a vaccine candidate in form of a protein, mRNA or DNA of recombinant Topo IIβKENV-HIV as an immunogen along with development of anti-HIV antibodies for protecting against HIV-1 infection in humans.

Another objective of our present invention is vaccination by protein, mRNA or DNA of conserved peptides with and without spacer derived from recombinant TopoIIβKENV-HIV as potent immunogens along with development of anti-HIV antibodies for protecting against HIV-1 infection in humans.

Another objective of our present invention is to use recombinant Topo IIβKENV-HIV as an inhibitor of HIV-1 replication targeted to HIV-1 entry.

Another objective of our present invention is to use recombinant Topo IIβKENV-HIV as a target for development of molecular inhibitors and siRNAs, antisense oligos for blocking HIV-1 entry, reverse transcription and replication.

Another objective of our present invention is use of protein, mRNA or DNA of recombinant Topo IIβKENV-HIV as a marker for HIV detection in HIV infected in humans.

### SUMMARY OF THE INVENTION

The following summary is provided to facilitate a clear understanding of the new features in the disclosed embodiment, and it is not intended to be a full, detailed description. A detailed description of all the aspects of the disclosed invention can be understood by reviewing the full specification, the drawing and the claims and the abstract, as a whole.

The present invention relates the rec-TopoIIβKENV-HIV, a N-terminal 196 amino acid region of HIV envelop protein gp120. Initially, in our present invention, it is cloned it in bacterial and mammalian expression vectors. After confirmation of the proteins from both the sources by SDS PAGE and western blot, the thermal stability of the Bac-rec-TopoIIβK_{ENV-HIV} protein is checked, and it is seen to possess high thermal stability and less aggregate formation. The binding capacity of the rec-TopoIIβKENV-HIV protein obtained from BL21 and HEK cells to bind to cell surface receptors of TZMb1 cells is also analysed. The specificity of these proteins towards CD4 receptors have also been seen.

The comparative binding of the Mam-rec-TopoIIβKENV-HIVprotein with that of Sim4 antibody, to CD4 receptor is done and it is seen that the presence of Sim4 slightly affects the binding of our protein of interest to CD4, whereas the presence of Mam-rec-TopoIIβK_{ENV-HIV} blocks the binding of Sim4 to CD4 up to 90%.

Further antiviral assay conducted with this rec-TopoIIβK_{ENV-HIV} protein shows significant decrease in viral p24 and hence the infection level in case of Mam-rec-TopoIIβKENV-HIV expressed in HEK, the change is quite subtle in case of Bac-rec-TopoIIβKENV-HIV. Thus rec-TopoIIβKENV-HIV protein can be used as an immunogen in HIV vaccines.

### BRIEF DESCRIPTION OF THE DRAWINGS

The manner in which the present invention is formulated is given a more particular description below, briefly summarized above, may be had by reference to the components, some of which is illustrated in the appended drawing It is to be noted; however, that the appended drawing illustrates only typical embodiments of this invention and are therefore should not be considered limiting of its scope, for the system may admit to other equally effective embodiments.

Throughout the drawings, the same drawing reference numerals will be understood to refer to the same elements and features.

The features and advantages of the present invention will become more apparent from the following detailed description a long with the accompanying figures, which forms a part of this application and in which:

FIG. 1: Diagram showing the Topoisomerase II beta kinase (TopoIIβKENV-HIV), a 196 amino acid coding nucleotide cloned into pEGFPC1 vector (FIG. 1a) and expressed in HEK cells (FIG. 1b) post transfection, in accordance with our present invention;

FIG. 2: Diagram showing the immunoreactivity of the recombinant TopoIIβKENV-HIV with (a) ID6 antibody and (b) 4G10 antibody, in accordance with our present invention;

FIG. 3: Diagram showing the inhibition of HIV-1 entry by the recombinant TopoIIβKENV, in accordance with our present invention;

FIG. 4: Diagram showing the Inhibition of HIV-1 replication by recombinant Topo IIβKENV-HIV, in accordance with our present invention;

FIG. 5: Diagram showing the Cloning and expression of Bac-rec-TopoIIβKENV-HIV, in accordance with our present invention;

FIG. 6: Diagram showing the Thermal stability plots for Bac-rec-TopoIIβKENV-HIV, in accordance with our present invention;

FIG. 7: Diagram showing the Binding assay of Bac-rec-TopoIIβKENV-HIV to CD4 receptors on TZMb1 cells, in accordance with our present invention;

FIG. 8: Diagram showing the Binding study of Bac-rec-TopoIIβKENV-HIV protein to CD4 receptors on TZMb1 cells, in accordance with our present invention;

FIG. 9: Diagram showing the Cloning and expression of Mam-rec-TopoIIβKENV-HIV protein, in accordance with our present invention;

FIG. 10: Diagram showing the Binding assay of Mam-rec-TopoIIβKENV-HIV to TZMb1 cells, in accordance with our present invention;

FIG. 11: Diagram showing the Binding of Sim4 to cell surface receptors on TZmb1 cells, in accordance with our present invention;

FIG. 12: Diagram showing the Competitive binding of Sim4 antibody and Mam-rec-TopoIIβKENV-HIV to CD4 receptors on TZMb1 cell, in accordance with our present invention;

FIG. 13: Diagram showing the HIV-1 replication p24 ELISA showing decrease in the infection level on addition of serum obtained from mice immunized with single booster of bacterial and mammalian expressed rec-TopoIIβKENV-HIV proteins, in accordance with our present invention.

FIG. 14: Diagram showing the regions of gp120 conserved among different subtypes of HIV-1 deposited in the Los Alamos database, in accordance with our present invention.

FIG. 15: Diagram showing selected regions from the conserved sequences has been linked using bridging peptides as spacers to create smaller peptide stretches with distinct conformation as shown, in accordance with our present invention.

FIG. 16: Diagram showing the interaction of the peptide constructs with CD4 receptors, in accordance with our present invention.

### DETAILED DESCRIPTION OF THE INVENTION

The principles of operation, design configurations and evaluation values in these non-limiting examples can be varied and are merely cited to illustrate at least one embodiment of the invention, without limiting the scope thereof.

The embodiments disclosed herein can be expressed in different forms and should not be considered as limited to the listed embodiments in the disclosed invention. The various embodiments outlined in the subsequent sections are constructed such that it provides a complete and a thorough understanding of the disclosed invention, by clearly describing the scope of the invention, for those skilled in the art.

Throughout this specification various indications have been given as to preferred and alternative embodiments of the invention. It should be understood that it is the appended claims, including all equivalents, which are intended to define the spirit and scope of this invention.

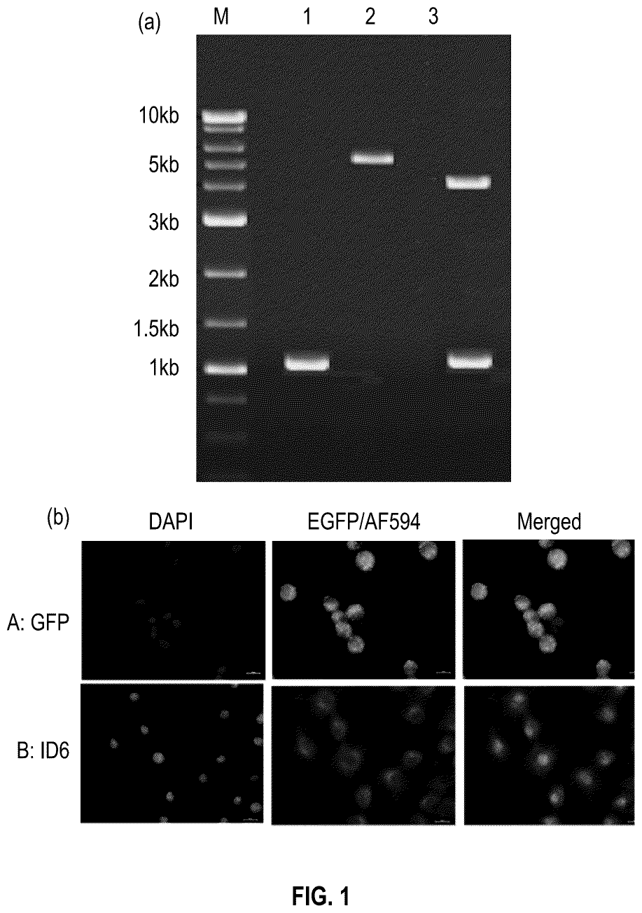

The present invention relates to the protein recombinant TopoIIβKENV-HIV which can be used as an immunogen in HIV vaccine. The current embodiment of the present invention relates to cloning and expression of recombinant rec-Topo IIBKENV-HIV (FIG. 1).

Mammalian Expression of Topoisomerase II beta kinase

Topoisomerase II beta kinase (TopoIIβKENV-HIV), a 196 amino acid coding nucleotide was cloned into pEGFPC1 vector (FIG. 1A) where Lane 1: Insert, Lane 2: Simple digest, Lane 3: Double digest and M: Marker, and expressed in HEK cells (FIG. 1B).

### Immunological Characterization of Rec-TopoIIβKENV-HIV

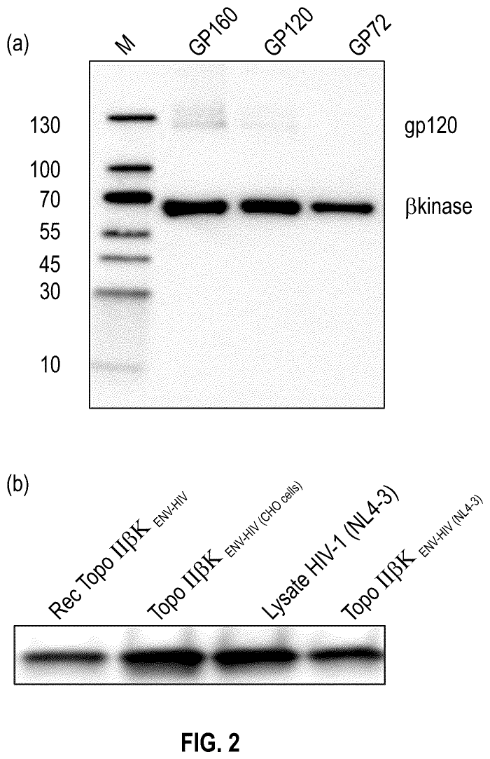

Expressed TopoIIβKENV-HIV was analyzed using gp120 monoclonal antibody ID6, which recognizes N-terminal 204-amino acids of gp 120. The results in FIG. 2A confirms that the recombinant TopoIIβKENV-HIV is recognized by ID6, further the immunoreactivity of recombinant TopoIIβKENV-HIV is conserved with that present in HIV-1 virus lysate and expressed in CHOJR-FL cells suggesting the recombinant TopoIIβKENV-HIV expressed in HEK cells retains immunological features of native protein.

Recombinant-TopoIIβKENV-HIV Inhibits HIV-1 Entry

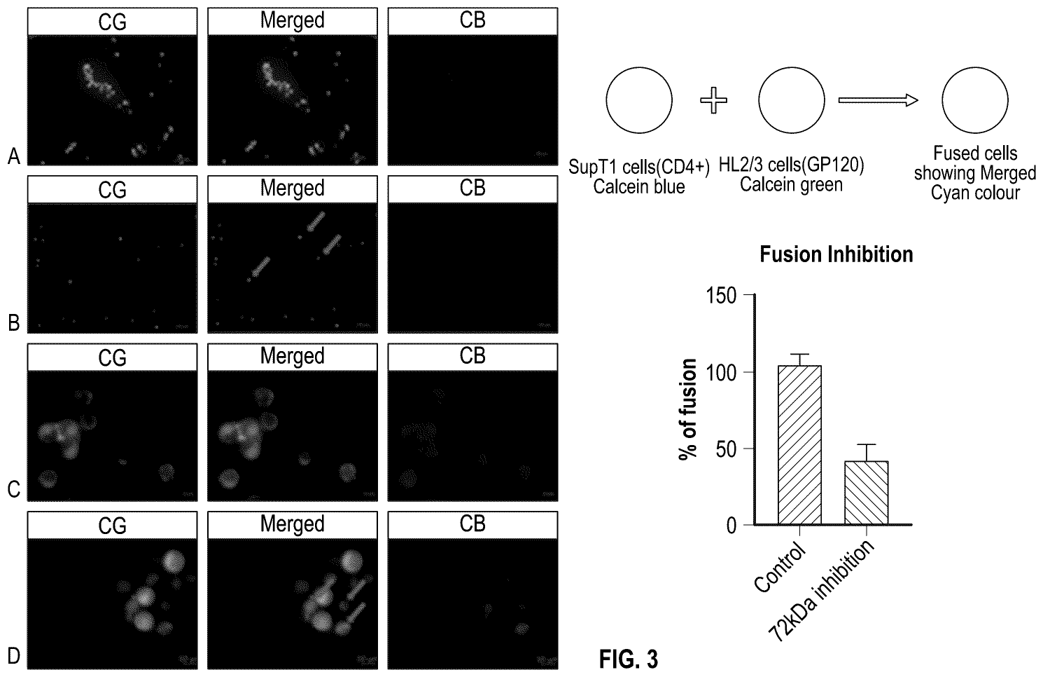

Action of rec-TopoIIβKENV-HIV on gp 120 mediated cell fusion of T cells, the results in FIG. 3 show that the TopoIIβKENV-HIV inhibits fusion of gp120 with cells, thus blocking viral entry (FIG. 3), wherein (A) shows control fusion using SupT1 (CD4+) cells in blue (CB) and HL2/3 (GP120+) cells in green (CG), (B) fusion experiment in the presence of 20 micrograms of purified 72KDa protein (C) 63× magnification of control fusion experiment and (D) 63× magnification of competitive fusion in the presence of TopoIIβKENV-HIV.

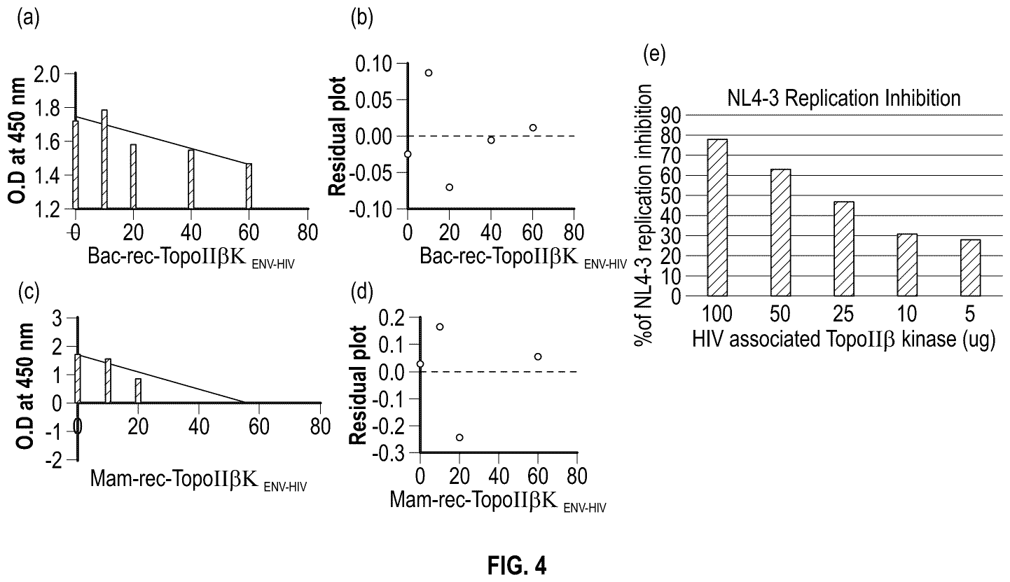

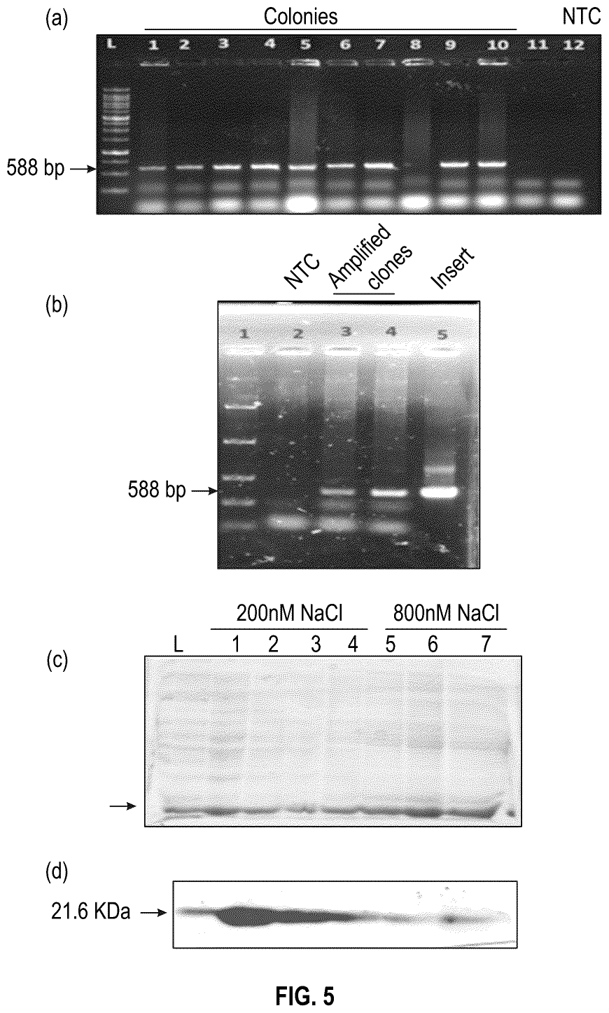

Rec-TopoIIβKENV-HIV neutralises HIV-1 HIV-1 infection was conducted in the presence of increasing concentrations of rec-TopoIIβKENV-HIV, the virus replicated on day 4 was estimated, the amount of virus replicated in the untreated cells was taken as negative control, the percent inhibition of virus replication was plotted against concentration of rec-TopoIIβKENV-HIV, the results in FIG. 4 show rec-TopoIIβKENV-HIV neutralises HIV-1. FIG. 4 shows the efficient virus neutralization activity of rec-TopoIIβKENV-HIV protein, when added to cells at different concentrations.

The bacterially expressed N-terminal 196 protein Bac-rec-TopoIIβKENV-HIV shows affinity towards CD4 receptors. For expression in bacteria, rec-TopoIIβKENV-HIV cloned in pET28 (a) vector. The clone was confirmed by colony PCR (b) and double digestion with the specific restriction enzymes. The clone was then transformed in BL21 cells and induced by 1 mM IPTG for protein expression, which was further purified using Ni/nta and MonoQ columns. The protein band has been confirmed by SDS PAGE and western blot with anti His antibody, this recombinant protein is termed as Bac-rec-TopoIIβKENV-HIV.

### Bac-Rec-TopoIIβKENV-HIV Protein High Thermal Stability:

The Nanotemper-prometheus NT. 48 was used to measure the thermal stability of the protein. 2-different concentrations of the Bac-rec-TopoIIβKENV-HIV protein (20 μM and 1 μM) have been used. The protein samples were loaded into the Nanotemper-prometheus NT. 48 with the help of capillaries, following a linear temperature ramp from 20° C. to 95° C., the transition of the protein from a folded to unfolded stage was recoded over time.

The dye-free technology helps monitor the minute alterations in the intrinsic fluorescence of a protein sample with change in temperature. The principle behind this assay states that after a certain temperature level protein starts to unfold from its quaternary to primary structure thus, it depicts the Tm of the protein which is the temperature at which the protein is 50% denatured, thus exposing the tryptophan residue. The aromatic side chains of these residues gives us the ratio of the fluorescence intensities of the protein at 350 nm and 330 nm, with respect to temperature.

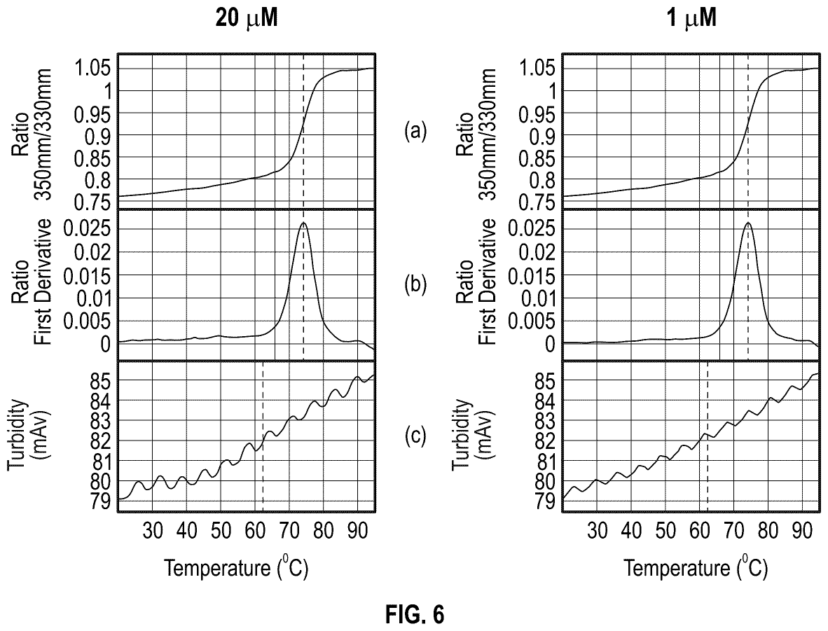

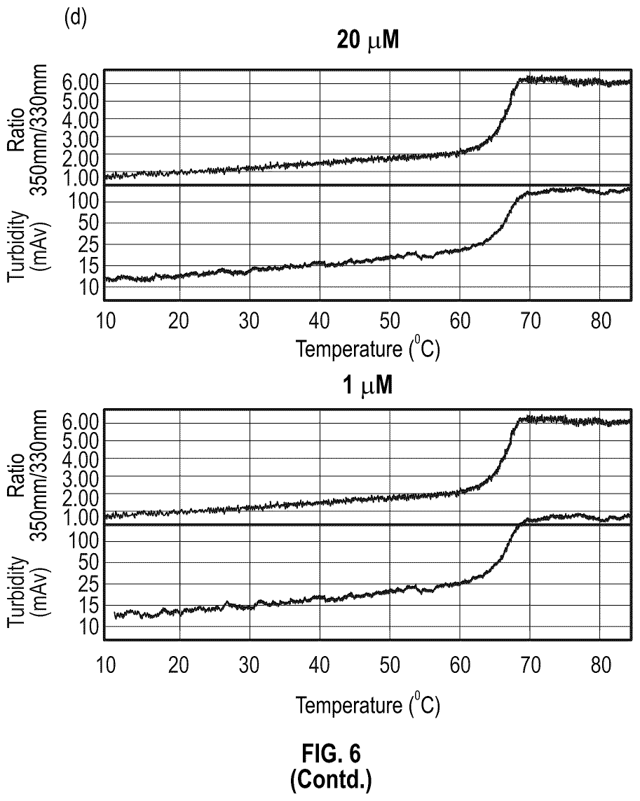

Two-different concentrations of the protein-20 and 1 μM have been used. The protein shows Tm at 75° C. The first derivative shows the range of temperature for which the transition spans. Here, a sharp peak depicts high thermal stability of the protein. The level of turbidity shows the temperature at which the protein aggregates. This is an important parameter since in general, misfolded proteins form aggregates and might lose its original functionality. The Nt196 shows the onset of turbidity only after 67° C. Thus it is also a quite stable protein in terms of aggregate formation, FIG. 6 where it shows the Bac-rec-TopoIIβKENV-HIV is highly stable at temperatures up to 75° C. A) The initial stretch shows the folded state of the protein, the slope depicts the transition of folded to unfolded state, and finally, the last part of the curve shows the un-folded state of the protein. B) The first derivative shows the range of temperature for which the transition spans from folded to unfolded state is short, and a sharp peak depicts higher thermal stability of the protein. C) This panel shows that the protein does not form aggregates easily, hence making it highly stable. D) A representation of the 350/330 ratio and the level of turbidity of the protein, depicting high thermal stability of the protein.

### Binding of Bac-Rec-TopoIIβKENV-HIV to Cell Surface Receptors of TZMb1 Cells:

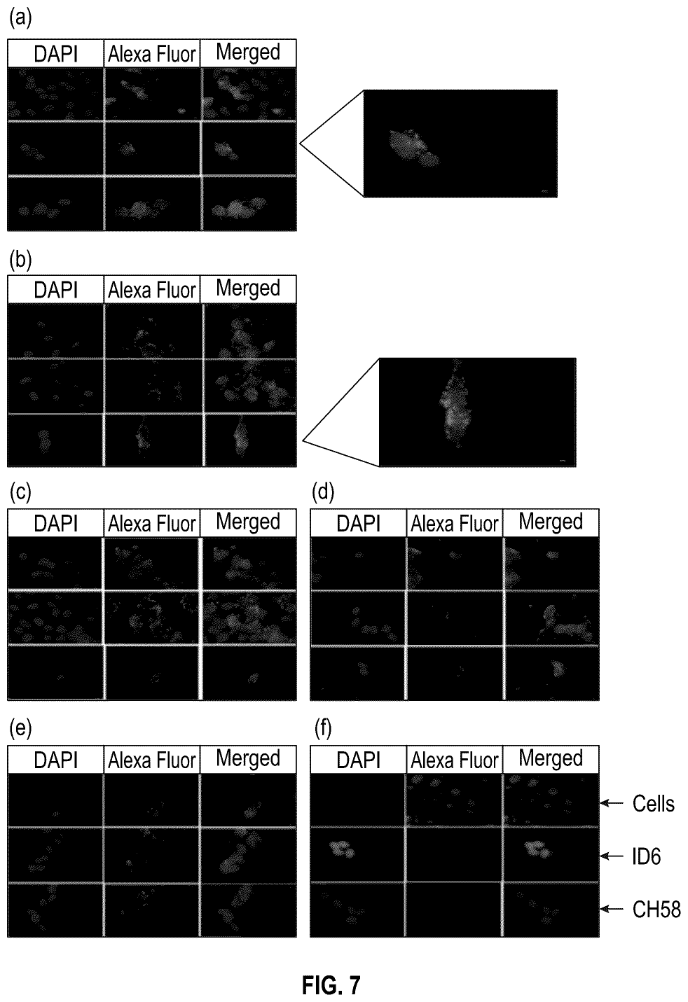

The bacterially expressed protein having affinity towards the cell surface receptors of TZMb1 cells can be detected by probing it with a primary antibody (ID6)-specific to the first 196 amino acids of HIV envelop, gp120 and in a different setup CH58 has been used as the primary antibody, which recognizes the V2 region of the HIV envelop. This was finally detected by a specific secondary antibody conjugated with Alexa Fluor 594. Hence the bound protein emitting red fluorescence was detected by fluorescence microscopy. The cell nucleus was stained with DAPI stain, and controls include cells with secondary antibody, cells with ID6 (primary antibody) and secondary antibody conjugated with Alexa Fluor 594, and cells with CH58 (primary antibody) and secondary antibody conjugated with Alexa Fluor 594, all without our protein of interest. The results are presented in FIG. 7. Five different concentrations of the Bac-rec-TopoIIβKENV-HIV protein has been used to check its ability to bind to the cell surface receptors on TZMb1 cells. A,B,C,D and E shows the interaction of the protein with cell surface receptors at 10,40,60,80, and 100 μg/ml concentrations respectively, which can be seen as red dots around the cells. In the first two panels in each of these figures ID6 had been used as the primary antibody, whereas in the third panel CH58 has been used. Secondary antibody is anti-mouse and is conjugated with Alexa Fluor 594 in each case. F) The first panel shows Control-1 with only cells and secondary antibody, second panel shows cells with primary antibody ID6 and secondary antibody, the third panel shows cells with primary antibody CH58 and secondary antibody.

### Specificity of Bac-Rec-TopoIIβKENV-HIV and CD4 Interaction:

If Bac-rec-TopoIIβKENV-HIV protein has affinity towards CD4 receptor, then on addition of soluble CD4, the bind to these soluble receptors, and will get washed away in the following steps. Thus, Bac-rec-TopoIIβKENV-HIV would not be available to bind to the cell surface CD4 receptors and hence no binding of primary and secondary antibodies, hence we might see a reduction in the signal intensity.

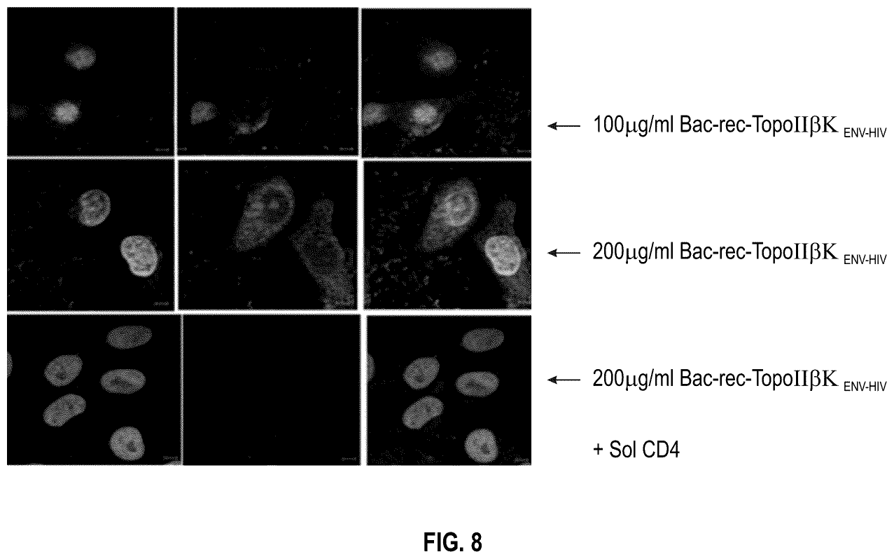

TZMb1 cells, having CD4 receptors were seeded in a 6-well plate, and was incubated with the Bac-rec-TopoIIβKENV-HIV at two-different concentrations, and in a separate well soluble CD4 was added to the cells along with Bac-rec-TopoIIβKENV-HIV in the ratio of 1:1. Following overnight incubation at 37° C., the cells with the proteins were probed with primary and secondary antibodies. Hoechst stain was used to stain the nucleus. Fluorescence microscopy images shows bright red signal in the presence of Bac-rec-TopoIIβKENV-HIV, which significantly reduces on addition of soluble CD4 along with our protein of interest as shown in FIG. 8. Affinity of Bac-rec-TopoIIβKENV-HIV to HIV-1 receptor CD4 was assessed by its binding to CD4 positive TZMBL cells, the results in FIG. 8 show a dose dependent binding of Bac-rec-TopoIIβKENV-HIV, further the specificity to CD4 was evaluated by competition of cells with soluble CD4, the results in FIG. 8 confirms that Bac-rec-TopoIIβKENV-HIV has specificity to CD4, thus effecting virus infection. In addition, an immunogen of Bac-rec-TopoIIβKENV-HIV would effectively neutralize the virus. Thus, it can serve as vaccine. In FIG. 8, Bac-rec-TopoIIβKENV-HIV binding to CD4. TZMb1 cells, having CD4 receptors were incubated with Bac-rec-TopoIIβKENV-HIV at 2-different concentrations (100 μg/ml and 200 μg/ml) in the first 2-panels. The third panel shows the effect of soluble CD4 protein on binding of Bac-rec-TopoIIβKENV-HIV to the cell surface CD4 receptors, when added together to the cells in the ratio of 1:1. Reduction in fluorescence in the presence of soluble CD4 shows that the protein has affinity and binds to soluble CD4. Hence, reduction in signal for cell surface protein.

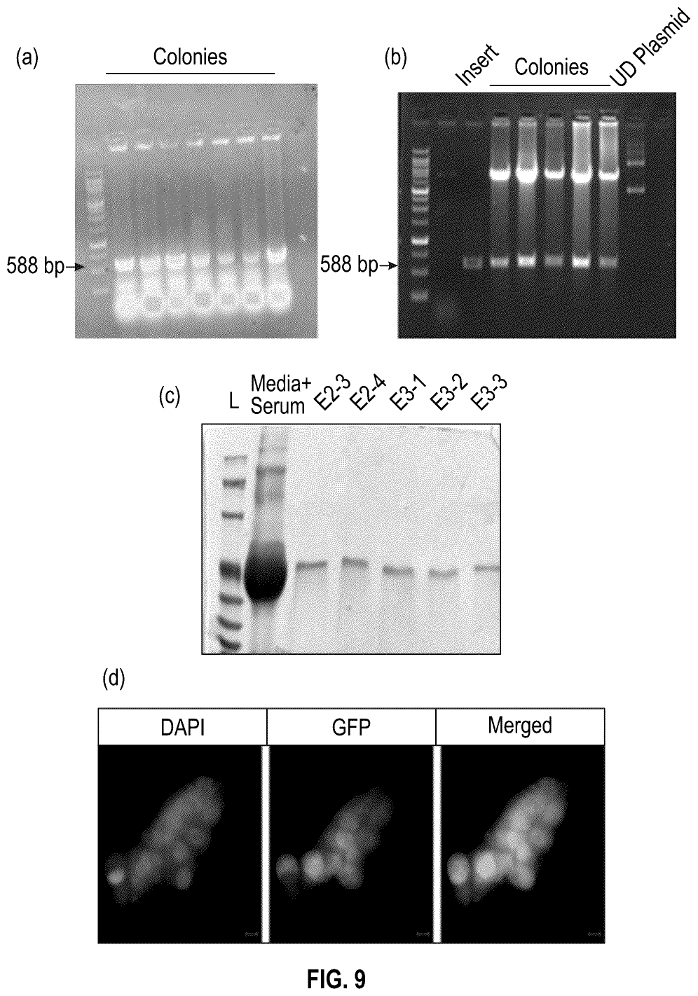

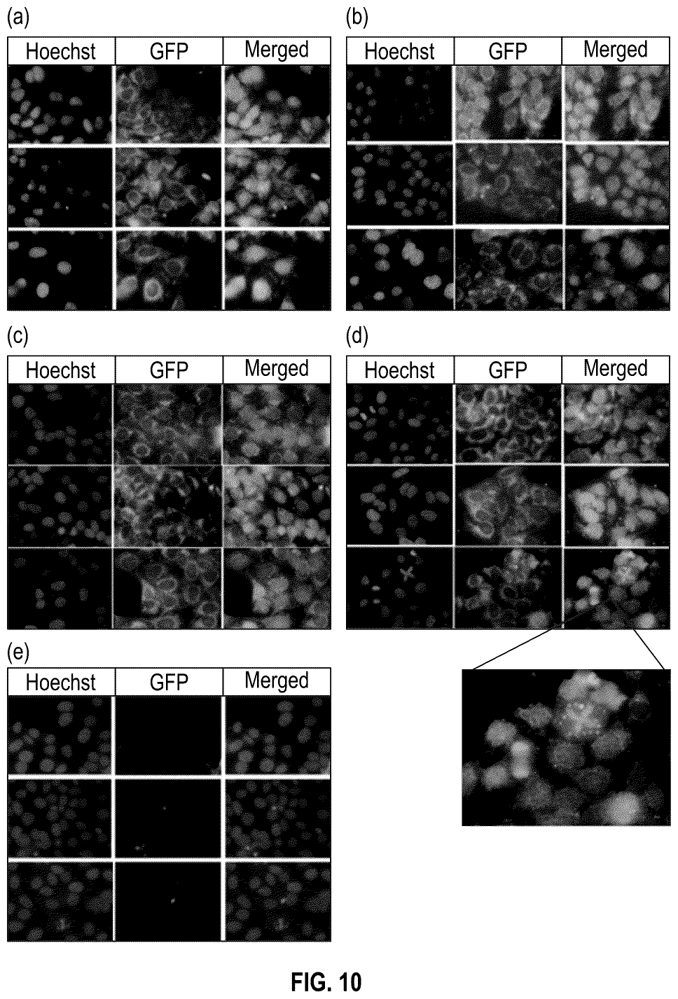

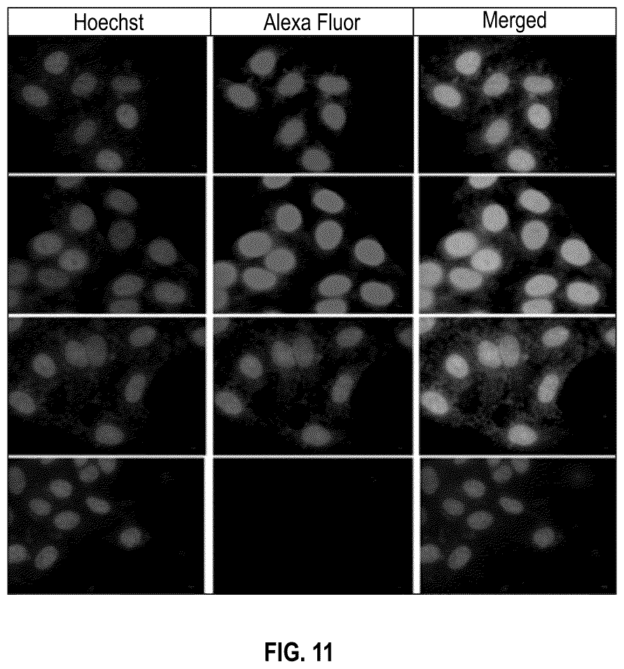

Mam-rec-TopoIIβKENV-HIV binds to cell surface receptors of TZMb1 cells. The Mam-rec-TopoIIβKENV-HIV was cloned in pEGFP-C1 and further transfected in HEK293T cells for protein expression (FIG. 9). The mammalian expressed recombinant protein Mam-rec-TopoIIβKENV-HIV being tagged with GFP was detected as green fluorescence signal by fluorescence microscopy (FIG. 10) after incubation with TZMb1 cells. The cells were fixed with 4% PFA, and cell nucleus was stained with Hoechst stain. In FIG. 9, it is shown that Mam-rec-TopoIIβKENV-HIV has been cloned in pEGFP-C1 vector. (A) The clone was confirmed with colony PCR (B) and by double digesting with EcoRI and SalI, (C) The protein was confirmed by SDS-PAGE. (D) Finally, the expression of the protein was confirmed by obtaining a fluorescence image of the HEK cells-transfected with Mam-rec-TopoIIβKENV-HIV protein.

In FIG. 10, four different concentrations of the rec-TopoIIβKENV-HIV protein (mammalian) has been used to check its ability to bind to the cell surface receptors on TZMb1 cells. A,B,C and D shows the interaction of the protein with cell surface receptors at 10,20,40, and 60 μg/ml concentrations respectively. The signal is visualized as bright green dots.E) Control having only cells.

Competitive binding between Sim4 and Mam-rec-TopoIIβKENV-HIV to CD4 receptors on TZMb1 cells.

Comparison of binding of Mam-rec-TopoIIβKENV-HIV in the presence of Sim4 antibody.

Sim4 is a CD4 specific antibody. Thus when TZMb1 cells having CD4 receptors are exposed to these proteins they can be detected by probing with anti-mouse secondary antibody, conjugated with Aexa Fluor 594, FIG. 11. In FIG. 11, TZMb1 cells were incubated with Sim4 for 3 hours, following washing with 1×PBS cells were fixed with 4% PFA, anti-mouse secondary antibody conjugated with Alexa Fluor was used for detection and the nucleus was stained with Hoechst stain. In control, cells are probed with secondary antibody, without Sim4. Sim4 shows binding to the cell surface receptors on TZMb1 cells at 1:20 dilution. Bound Sim4 is detected as bright red signal. In this assay, TZMb1 cells have been primarily incubated with Sim4 antibody for 3 hours, which would block the CD4 receptor on the cell surface, thus making it less available for Mam-rec-TopoIIβKENV-HIVto bind with, which was further added to these cells and incubated for another 3 hours. This was followed by cell fixation with 4% paraformaldehyde (PFA), and probing with anti-mouse secondary antibody conjugated with Alexa Fluor 594. Mam-rec-TopoIIβKENV-HIVbeing tagged with GFP gives green fluorescence. In the first case there is a strong signal for Sim4 (red) and a comparatively decreased signal of Mam-rec-TopoIIβKENV-HIV (green), and in the merged image we see yellow fluorescence as a combination of both the proteins.

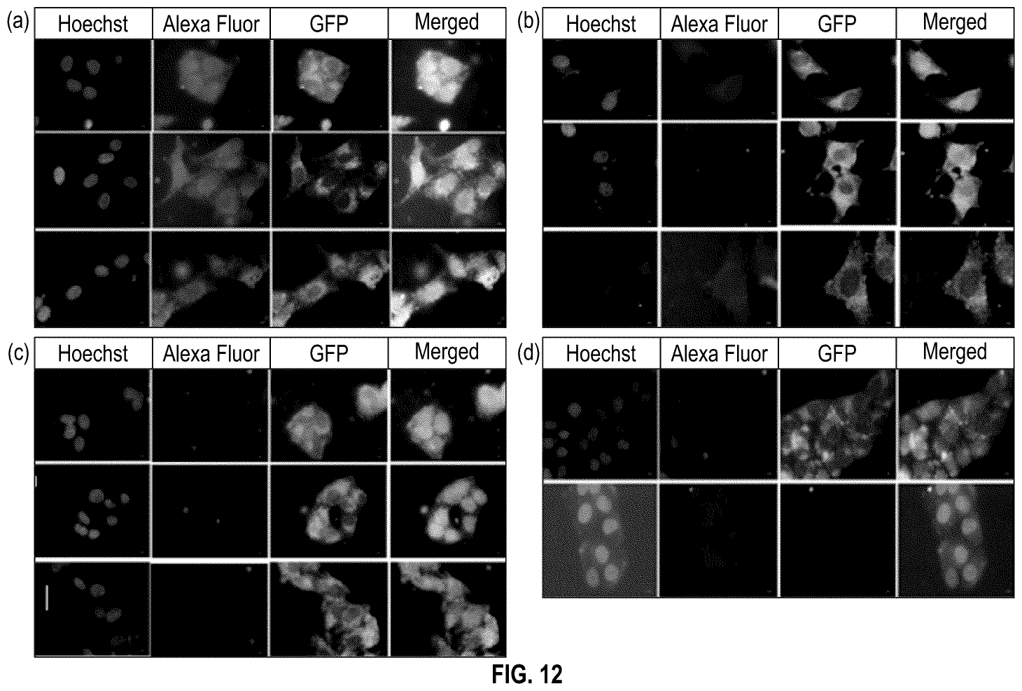

In the second case, where Mam-rec-TopoIIβKENV-HIVwas added prior to Sim4, the signal for Sim4 is seen to be significantly decreased. In the third case, where both the proteins were added together we find a similar result as that of the second scenario, suggesting that Mam-rec-TopoIIβKENV-HIVhas higher affinity to the cell surface receptors (CD4) on TZMb1 cells as compared to Sim4. This also suggests that our protein of interest specifically targets and binds to the CD4 receptors on the cell surface, FIG. 12.

For control-1, TZMb1 cells were probed with secondary antibody conjugated with Alexa Fluor 594 without the presence of Mam-rec-TopoIIβKENV-HIVor Sim4, and for control-2, cells were incubated with Mam-rec-TopoIIβKENV-HIV and probed with the secondary antibody conjugated with Alexa Fluor 594.

In FIG. 12, in order to check the affinity of the Mam-rec-TopoIIβKENV-HIVtowards CD4 receptor TZMb1 cells having CD4 receptors were first incubated with Sim4 antibody (antibody specific to CD4 receptor) followed by incubation with Mam-rec-TopoIIβKENV-HIVprotein tagged with GFP. Addition of secondary antibody conjugated with Alexa Fluor 594 gives signal for Sim4, whereas green fluorescence signals for Mam-rec-TopoIIβKENV-HIV protein. A) First lane shows the nucleus stained with Hoechst stain, second lane shows bound Sim4 in red, third lane shows the bound Mam-rec-TopoIIβKENV-HIVprotein tagged with GFP, thus can be detected by the green fluorescence, and finally, the fourth lane shows a merged image, where binding of Sim4 (red) and Mam-rec-TopoIIβKENV-HIV (green) gives an yellow fluorescence. B) Here the addition of Mam-rec-TopoIIβKENV-HIVprotein prior to Sim4 shows a marked decrease in the signal intensity for Sim4 as compared to rec-TopoIIβKENV-HIV (mammalian). C) When added together, we see a decrease in Sim4 signal with respect to the signal for Mam-rec-TopoIIβKENV-HIV protein. D) Shows control-1 (Mam-rec-TopoIIβKENV-HIV added to the cells and probed with secondary antibody conjugated with Alexa Fluor 594). Panel 2 shows control-2 (cells probed with secondary antibody conjugated with Alexa Fluor 594), without the recombinant protein of interest.

The antiviral assay shows that both the bacterial and mammalian rec-TopoIIβKENV-HIV proteins lowers the level of infection in SupT1 cells (FIG. 4).

In order to check the antiviral activity of the Bac-rec-TopoIIβKENV-HIV protein, a p24 ELISA was conducted. Briefly, SupT1 cells were incubated for 3 hours with rec-TopoIIβKENV-HIV protein-expressed in bacterial and mammalian cells. NL4-3 (subtype B) was used to infect these cells in a serum free media. Serum was added after 4 hours of infection, and post 4-days virus was estimated using p24 assay. Both bacterial and mammalian proteins show significant better antiviral response as compared to the bacterially expressed protein, FIG. 4, SupT1 cells having the receptor-CD4 and co-receptors-CXCR4 and CCR5 allows it to be efficiently targeted by HIV. Hence SupT1 cells were incubated with the bacterially and mammalian expressed proteins, individually for 3 hours. These cells were further subjected to infection by NL4-3 for 48 hours. Following which p24 ELISA was conducted, and absorbance was taken at 450 nm wavelength. In FIG. 4., a) The rec-TopoIIβKENV-HIV protein expressed in bacteria shows slight decrease in the level of infection. b) Shows the residual plot for the same. c) The rec-TopoIIβKENV-HIV protein expressed in HEK cells shows significant level of reduction in the infection level as compared to the bacterially expressed protein. d) Shows the residual plot for the mammalian expressed protein. e) shows inhibition of NL4-3 replication in presence of HIV derived TopoIIβ Kinase.

Antiviral activity of the serum collected from mice immunized with Bac-rec-TopoIIβKENV-HIV and Mam-rec-TopoIIβKENV-HIV proteins respectively.

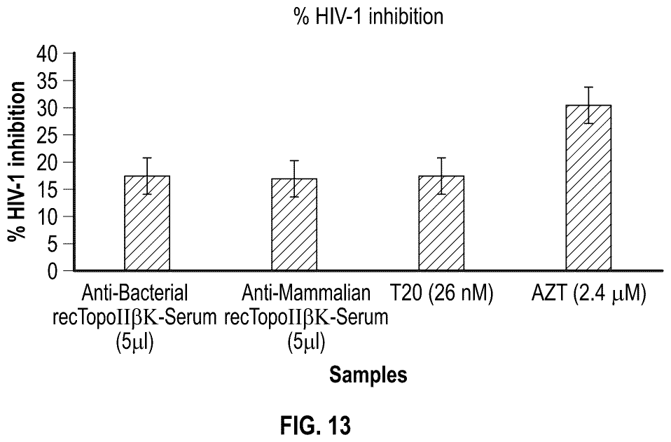

To check the immunogenicity of the rec-TopoIIβKENV-HIV proteins from bacterial and mammalian cells, female BALB/c mice of 8-10 weeks were used. Following acclimatizing the mice for 1-week, they were immunized with 50 μg of Bac-rec-TopoIIβKENV-HIV and Mam-rec-TopoIIβKENV-HIV proteins separately. The first dose consisted of 50 μg of the respective proteins, 150 μl of PBS and 150 μl of Freund's complete adjuvant. The same dose dose was repeated as the first booster after 21 days of first dose. 200 μl of blood was collected into microcentrifuge tubes (MCTs) from the retro-orbital vein of the mice. The tubes were allowed to rest for 1.5 hours, and serum was collected into fresh MCTs. Dot blot was performed to check the presence of specific antibody in the serum sample. 5 μl of each of these serum samples were added separately to NL4-3 virus, and following incubation for 1 hour at 37° C. and 5% CO2 this viral-serum mixture was added to TZMb1 cells. The viral supernatant was used to perform p24 ELISA. The rationale behind this is-TZMb1 cells having CD4 receptors on its surface would be infected by the virus, whereas on addition of the respective serum samples, having the potential antibodies against the recombinant proteins, should bind to the viral gp120, and on addition to cells, the vital epitope-already being occupied with the specific antibodies, would be unavailable to bind to CD4 receptors. Hence, there should be decrease in the infection rate. Zidovudine (AZT) and Enfuvirtide (T20) has been used as positive controls, negative control is devoid of any drug or serum, and blank contains only cells (FIG. 13). The serum after first booster showed the presence of anybody through dot blot, while significant activity is observed. Further, dose-titration would enhance higher antibody response.

Interaction of the conserved regions of gp120 with cell surface CD4 receptors. HIV envelope protein plays a major role in attachment and entry of the virus into the cell. The high amount of variability the viral envelope protein, gp120 poses a challenge in the field of vaccine development against HIV. Apart from the several variable regions, the viral envelope protein also possesses certain conserved sequences, necessary for attachment and entry into the cell. Thus, in our study, our major focus will be these conserved regions which might act as a potential vaccine candidate against HIV.

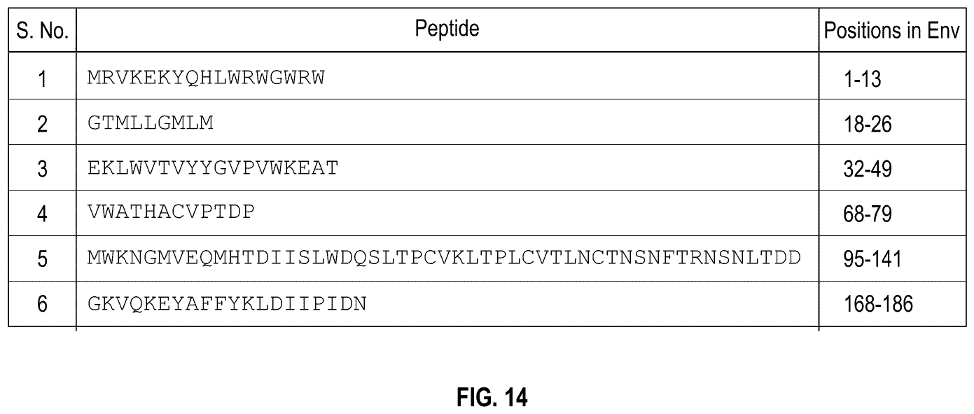

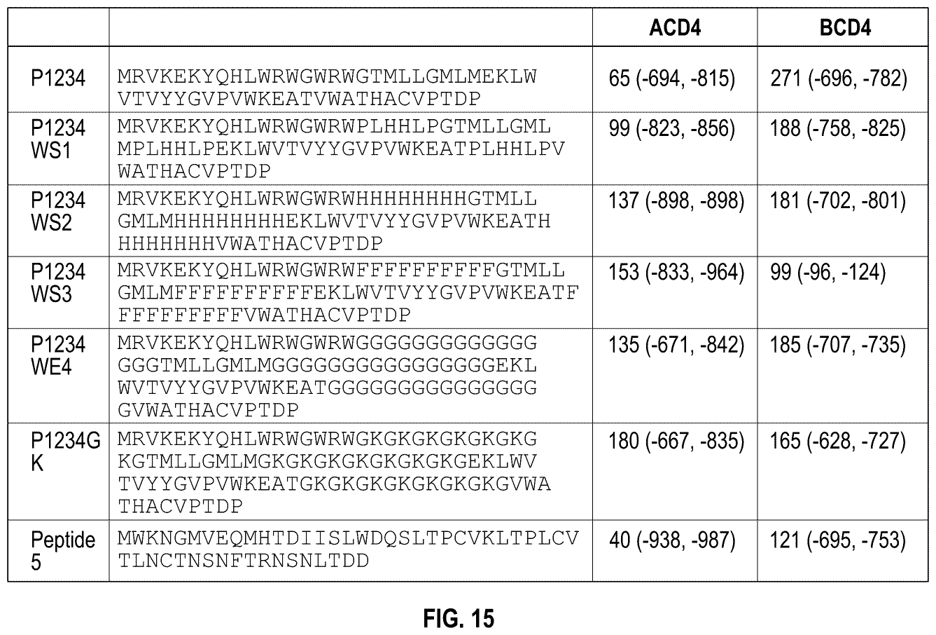

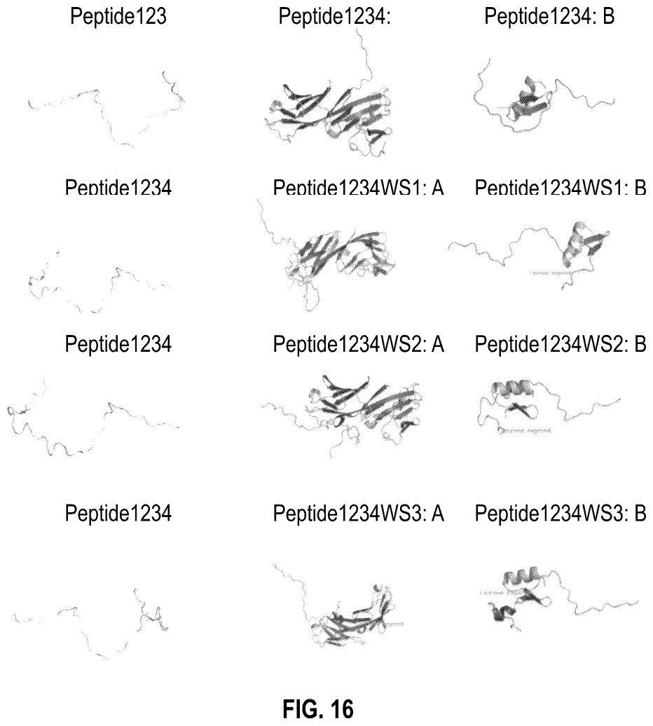

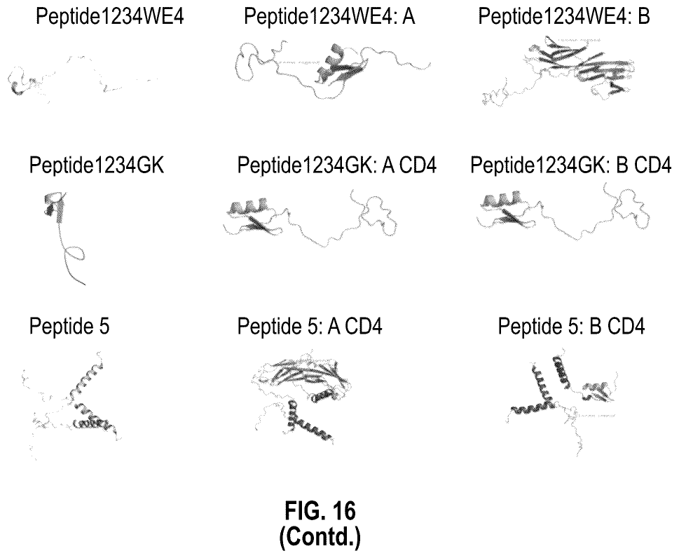

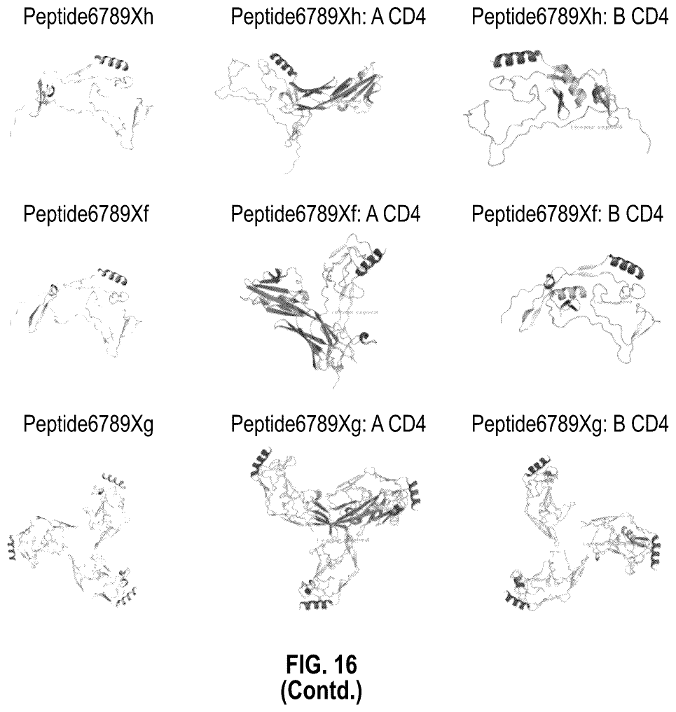

The conserved sequences have been found within the sequence of rec-TopoIIβKENV-HIV amino acid sequences from the Los Alamos website. Among which, 6 regions have been found to be conserved among different subtypes of HIV-1 (FIG. 14). These regions have been connected into smaller fragments using certain bridging peptides (FIG. 15), and their interaction with CD4 has been analysed (FIG. 16). Briefly, model has been developed with swis prot, optimized with molecular dynamics following docking to CD4 using Cluspro. In the drawing A: CD4 is CD4 full-length structure and B: CD4 is the CD4 portion exposed with envelope binding region obtained from PDB.

### Methods:

### Cloning and Expression

The gene of interest was PCR amplified from pNL4-3 plasmid, which was further ligated in pET 28 (a) and pEGFP-C1 vectors for bacterial and mammalian expression respectively, and transformed in DH10B cells. Plasmid was isolated from these transformed cells using GeneJET mini-prep kit by Thermo Scientific. The plasmid concentration was checked in nanodrop. The clones were confirmed by double digesting the clones with NdeI-SalI and EcoRI-SalI for bacterial and mammalian clones respectively, and running on 0.7% agarose gel.

Protein expression and purification rec-TopoIIβKENV-HIV (bacterial) The confirmed clone was further transformed in BL21 cells. Following inducing protein expression by 1 mM IPTG for 4.5 hours, cells were harvested by centrifuging at 10,000 rpm for 10 minutes at 4° C. The cell pellet was resuspended in lysis buffer-containing 500 mM NaCl, 50 mM Tris HCL and 1 mM PMSF, and was sonicated at 35 amp for 45 seconds, with 1 minute breaks. This sonicated product was further centrifuged for 30 minutes to remove the cell debris. The collected supernatant was loaded to Ni/nta column for purification. MonoQ column has been used for further removal of non-specific bands. The protein was further confirmed by running the sample on 15% SDS PAGE, and western blot with anti His antibody, where the protein showed band at 21.6 KDa.

### Rec-TopoIIβKENV-HIV (Mammalian)

The confirmed clone was directly used for transfecting HEK cells, mediated by Lipofectamine 3000. A serum free media was used for transfection, and after 25 hours the media was collected from these transfected cells, which was directly loaded onto MonoQ column after equilibrating the column with 20 mM Tris HCL of pH 8.6. The protein was confirmed by running on 10% SDS PAGE. HEK was seeded on coverslips in 6-well palte, and was transfected with 2 μg of plasmid (196 cloned in pEGFP-C1). These cells were fixed with 4% PFA for 20 minutes, and after washing the cells with 1×PBS the cells were stained with DAPI and following PBS wash cells were mounted on 30% glycerol and was imaged by fluorescence microscope.

### Model Development and Docking

Model for the conserved peptide were developed with swis prot, optimized with molecular dynamics, further it was followed by docking to CD4 using Cluspro.

### Cell Culture

SupT1 cells were propagated in RPMI-1640, CHO k1, CHO JRFL gp160, CHO NL4-3 gp160, TZMb1 and HEK 293T were propagated in DMEM-F12 (Gibco, Invitrogen, CA, USA) with 10% FBS (Gibco) maintaining standard conditions of 5% CO2 and 37° C.

Fusion assay: Surface Gp160 expressing HL2/3 cells and CD4 and co-receptor expressing SupT1 cells has been loaded with different fluorescent-ester dyes and co-incubated. The receptor mediated fusion of these two cells would be followed by the presence of both fluorescence signals within the same cell, which could be imaged. The presence of fusion inhibitory drugs (kinase inhibitors) would inhibit this fusion and thereby the cell population with both fluorescence signals would be lesser in population. CD4 and CCR5 & CXCR4 expressing SupT1 cells will be loaded with 20 μM calcein blue dye and the gp 120-41 expressing HL2/3 cells will be loaded with 0.5 μM calcein green AM, and the cells will be incubated at 37° C. for 1 hour, in a 5% CO2 humidified incubator. Post incubation, the cells will be washed with PBS, fresh media will be added and the HeLa (HL 2/3) cells will be incubated with 50 nM T20 (enfuvirtide), 50 μM of Drugs A, B, C and F, and incubated for 1 hour. The cells will be then mixed (10,000:30,000 of SupT1: HL 2/3) and co-cultured for 2 hours. After co-culturing, the cells will be mounted on a poly-D-lysine coated cover slip and the cells will be observed for the fluorescent signals on a trinocular fluorescent microscope. The cells will be checked for both calcein blue, AM (360 nm/449 nm) and calcein green, AM (493 nm/513 nm) signals.

Viral Infection: 5×106 cells were infected with HIV-1 subtype B (NL4-3) and was collected 4-days post infection. 20 ng (p24 quantity) of virus for every 2×106 cells (˜100 MOI) was used in serum free media, 4 hours post-infection 10% serum was added to allow cell growth. All infections were carried out in SupT1 cells only.

### Viral Infectivity (p24 Assay)

After 4-days of infection, the virus quantity was measured in the cell culture supernatants by estimating the viral p24 protein with p24 ELISA kit following the manufacturer's instructions. Briefly 100 μL of cell culture supernatant was added to the ELISA plate wells containing 25 μL of disruption buffer and incubated at 37° C. After 1 hour of incubation, these wells were washed three times. Then 100 μL of anti-HIV-1 p24 peroxidase-conjugated antibody was added and incubated at 37° C. After 1 hour, ELISA plate wells were washed, 100 μL of peroxidase substrate solution was added and incubated in dark at room temperature for 20 minutes. The reaction will be stopped by addition of 100 μL stop solution or 2N H2SO4 and the color intensity was measured at 450 nm. Un-infected cell culture supernatant or complete propagation media was used as negative control.

### Mice Immunization

Female BALB/c mice of 8-10 weeks were used in our experiment. Following acclimatizing the mice for 1-week, they were subcutaneously injected with 50 μg of Bac-rec-TopoIIβKENV-HIV and Mam-rec-TopoIIβKENV-HIV proteins separately. The first dose consisted of 50 μg of the respective proteins, 150 μl of PBS and 150 μl of Freund's complete adjuvant. The same dose dose was repeated as the first booster after 21 days of first dose, except for that 150 μl of Freund's incomplete adjuvant was used. The weight of the mice was regularly monitored pst immunization.

Nucleotide sequence
Sequence ID-1
Nucleic acid sequence of bac-recombinant HIV-1
derived Topoisomerase II beta kinase
cacttgtggagatgggggtggaaatggggcaccatgctccttgggatat
tgatgatctgtagtgctacagaaaaattgtgggtcacagtctattatgg
ggtacctgtgtggaaggaagcaaccaccactctattttgtgcatcagat
gctaaagcatatgatacagaggtacataatgtttgggccacacatgcct
gtgtacccacagaccccaacccacaagaagtagtattggtaaatgtgac
agaaaattttaacatgtggaaaaatgacatggtagaacagatgcatgag
gatataatcagtttatgggatcaaagcctaaagccatgtgtaaaattaa
ccccactctgtgttagtttaaagtgcactgatttgaagaatgatactaa
taccaatagtagtagcgggagaatgataatggagaaaggagagataaaa
aactgctctttcaatatcagcacaagcataagagataaggtgcagaaag
aatatgcattcttttataaacttgatatagtaccaatagataataccag
ctataggttgataagttgtaacacctcagtcattacacaggcctgtcca
Nucleic acid sequence of mam-recombinant HIV-1
derived Topoisomerase II beta kinase
Sequence ID-2
cacttgtggagatgggggtggaaatggggcaccatgctccttgggatat
tgatgatctgtagtgctacagaaaaattgtgggtcacagtctattatgg
ggtacctgtgtggaaggaagcaaccaccactctattttgtgcatcagat
gctaaagcatatgatacagaggtacataatgtttgggccacacatgcct
gtgtacccacagaccccaacccacaagaagtagtattggtaaatgtgac
agaaaattttaacatgtggaaaaatgacatggtagaacagatgcatgag
gatataatcagtttatgggatcaaagcctaaagccatgtgtaaaattaa
ccccactctgtgttagtttaaagtgcactgatttgaagaatgatactaa
taccaatagtagtagcgggagaatgataatggagaaaggagagataaaa
aactgctctttcaatatcagcacaagcataagagataaggtgcagaaag
aatatgcattcttttataaacttgatatagtaccaatagataataccag
ctataggttgataagttgtaacacctcagtcattacacaggcctgtcca
Amino Acid Sequence: (196 amino acids)
Sequence ID-3
HLWRWGWKWGTMLLGILMICSATEKLWVTVYYGVPVWKEATTTLFCASD
AKAYDTEVHNVWATHACVPTDPNPQEVVLVNVTENFNMWKNDMVEQMHE
DIISLWDQSLKPCVKLTPLCVSLKCTDLKNDTNTNSSSGRMIMEKGEIK
NCSFNISTSIRDKVQKEYAFFYKLDIVPIDNTSYRLISCNTSVITQACP
Nucleic acid sequence (Mutant 196 amino acids)
Sequence ID-4
caGCAgtggagatgggggtggaaatggggcaccatgctccttgggatat
tgatgatctgtagtgctacagaaaaattgtgggtcacagtctattatgg
ggtacctgtgtggaaggaagcaaccaccactctattttgtgcatcagat
gctaaagcatatgatacagaggtacataatgtttgggccacacatgcct
gtgtacccacagaccccaacccacaagaagtagtattggtaaatgtgac
agaaaattttaacatgtggaaaaatgacatggtagaacagatgcatgag
gatataatcagtttatgggatcaaagcctaaagccatgtgtaaaattaa
ccccactctgtgttagtttaaagtgcactgatttgaagaatgatactaa
taccaatagtagtagcgggagaatgataatggagaaaggagagataaaa
aactgctctttcaatatcagcacaagcataagagataaggtgcagaaag
aatatgcattcttttataaacttgatatagtaccaatagataataccag
ctataggttgataagttgtaacacctcagtcattacacaggcctgtcca
Amino Acid Sequence (Mutant): (196 amino acids)
Sequence ID-5
QQWRWGWKWGTMLLGILMICSATEKLWVTVYYGVPVWKEATTTLFCASD
AKAYDTEVHNVWATHACVPTDPNPQEVVLVNVTENFNMWKNDMVEQMHE
DIISLWDQSLKPCVKLTPLCVSLKCTDLKNDTNTNSSSGRMIMEKGEIK
NCSFNISTSIRDKVQKEYAFFYKLDIVPIDNTSYRLISCNTSVITQACP

The Applicant herewith submits the sequence listing file in XML format. Also a reference to the sequence listing is added to the specification. Please find below the sequence listing file details as required by 37 CFR 1.835 (a) (2) or 1.835 (b) (2);
1. The name of the XML file is “10-58-USUTIL-SL”
2. The date of creation is Feb. 13, 2024; and
3. The size of the XML file in bytes is “8000 bytes”

We claim:

1. A protein, mRNA and DNA of immunogenic recombinant HIV-derived Topoisomerase II beta kinase as an immunogen for HIV vaccine, wherein the Nucleotide sequence is given by:
cacttgtggagatgggggtggaaatggggcaccatgctccttgggatat
tgatgatctgtagtgctacagaaaaattgtgggtcacagtctattatgg
ggtacctgtgtggaaggaagcaaccaccactctattttgtgcatcagat
gctaaagcatatgatacagaggtacataatgtttgggccacacatgcct
gtgtacccacagaccccaacccacaagaagtagtattggtaaatgtgac
agaaaattttaacatgtggaaaaatgacatggtagaacagatgcatgag
gatataatcagtttatgggatcaaagcctaaagccatgtgtaaaattaa
ccccactctgtgttagtttaaagtgcactgatttgaagaatgatactaa
taccaatagtagtagcgggagaatgataatggagaaaggagagataaaa
aactgctctttcaatatcagcacaagcataagagataaggtgcagaaag
aatatgcattcttttataaacttgatatagtaccaatagataataccag
ctataggttgataagttgtaacacctcagtcattacacaggcctgtcca
And Amino Acid Sequence:
HLWRWGWKWGTMLLGILMICSATEKLWVTVYYGVPVWKEATTTLFCASD
AKAYDTEVHNVWATHACVPTDPNPQEVVLVNVTENFNMWKNDMVEQMHE
DIISLWDQSLKPCVKLTPLCVSLKCTDLKNDTNTNSSSGRMIMEKGEIK
NCSFNISTSIRDKVQKEYAFFYKLDIVPIDNTSYRLISCNTSVITQACP
